(19) United States
(12) Patent Application Publication
(10) Pub. No.: US 2024/0428893 A1
(43) Pub. Date: Dec. 26, 2024

(54) METHODS FOR ENHANCING COMPLETE DATA EXTRACTION OF DIA DATA

(71) Applicant: DH Technologies Development Pte. Ltd., Singapore (SG)

(72) Inventor: Stephan A. TATE, Barrie (CA)

(21) Appl. No.: 18/698,672

(22) PCT Filed: Oct. 5, 2022
(86) PCT No.: PCT/IB2022/059511

§ 371 Date: Apr. 4, 2024

Related U.S. Application Data
(60) Provisional application No. 63/262,112, filed on Oct. 5, 2021.

(51) Int. Cl. G16C 20/20; G16C 20/40; G16C 20/70

(52) U.S. Cl. CPC G16C 20/20; G16C 20/40; G16C 20/70

(57) ABSTRACT

The n spectra of a DIA method are compared to a library of product ion spectra to identify an initial i compounds corresponding to l spectra. A reinforcement learning algorithm (RLA) is performed. (a) An agent of the RLA performs an action Aₜ that includes searching one or more compound databases for compounds related to the i compounds, producing j related compounds, and applying one or more deep learning prediction algorithms to predict k spectra for the i+j compounds. (b) An environment of the RLA compares the k spectra to the n spectra, producing a state, Sₜ, in which i+j compounds produce m matching compounds and a reward, Rₜ, for the agent if m>i. (c) If the Rₜ is produced, the i compounds are set to the m compounds and the l spectra are set to the k spectra, and steps (a)-(c) are repeated.

### RELATED APPLICATIONS

This application claims the benefit of U.S. Provisional Patent Application Ser. No. 63/262,112, filed on Oct. 5, 2021, the content of which is incorporated by reference herein in its entirety.

### INTRODUCTION

The teachings herein relate to systems and methods for extracting additional information from a data-independent acquisition (DIA) mass spectrometry experiment. More particularly the teachings herein relate to systems and methods in which additional compounds are extracted from DIA data using a reinforcement learning algorithm in which related compounds of previously identified compounds are used to increase the number of compounds identified from the DIA data.

The systems and methods herein can be performed in conjunction with a processor, controller, or computer system, such as the computer system of FIG. 1.

### DIA Data Extraction

As described below, data-independent acquisition (DIA) is an untargeted and non-specific fragmentation method. In a traditional DIA method, the actions of the tandem mass spectrometer are not varied among MS/MS scans based on data acquired in a previous precursor or product ion scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

DIA data is very information-rich, and, in most cases, data processing is undertaken with the use of a spectral library. This library provides spectra of compounds that may be present within the sample and enable quantitative information to be extracted for them. Currently, if a compound is not present within the spectral library, then there is no solution to be able to extract the information from the DIA data. In other words, if a compound is not in the library it cannot be found in the DIA data.

Libraries that are used to extract information from DIA data files come from a range of different sources. They can come from multiple data-dependent acquisition (DDA) type of experiments, where product ion spectra are matched to different compounds and then the result is used to build a specific library. Also, in more recent cases, they can come from the prediction of peptide spectra through the use of deep learning methods.

The deep learning prediction methods such as ProSIT, pDeep3, or MS2PIP provide a method for the prediction of fragment pattern for product ion spectra as well as the retention times of the peptides through the use of internal calibration or through the use of tools such as DeepRT. In one exemplary case, MS2PIP has been used to generate proteome-wide libraries for all theoretical peptides that are then used to extract proteins or peptides from DIA data.

Two main problems have appeared when using deep learning prediction methods to extract proteins or peptides from DIA data. First, such methods can produce a mass space for the product ions that is crowded. This results in very large libraries with many peptides that are not accessible to mass spectrometry technology. As a result, this causes an increase in the false-negative rate and can, therefore, affect the overall false discovery rate (FDR) scoring of a real signal. This, in turn, diminishes the function of the expanded library. Secondly, the extremely large libraries increase the computational time as every compound needs to be extracted. In addition, when modifications to different sequences are taken into consideration, this required increase in computation time can become an intractable problem.

As a result, there is a need for systems and methods that can allow deep learning prediction methods to be used to extract information from DIA data without producing a large amount of false-negative results and without significantly increasing the computation time required.

### Tandem Mass Spectrometry Background

In general, tandem mass spectrometry, or mass spectrometry/mass spectrometry (MS/MS), is a well-known technique for analyzing compounds. Tandem mass spectrometry involves ionization of one or more compounds from a sample, selection of one or more precursor ions of the one or more compounds, fragmentation of the one or more precursor ions into fragment or product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. Three broad categories of these workflows are, targeted acquisition, information dependent acquisition (IDA) or data-dependent acquisition (DDA), and data-independent acquisition (DIA).

In a targeted acquisition method, one or more transitions of a precursor ion to a product ion are predefined for a compound of interest, or just the precursor mass is provided if a full fragmentation spectra is to be collected. As a sample is being introduced into the tandem mass spectrometer, the one or more transitions are interrogated during each time period or cycle of a plurality of time periods or cycles. In other words, the mass spectrometer selects and fragments the precursor ion of each transition and performs a targeted mass analysis for the product ion of the transition. As a result, an intensity (a product ion intensity) is produced for each transition. Targeted acquisition methods include, but are not limited to, multiple reaction monitoring (MRM) and selected reaction monitoring (SRM).

In an IDA method, a user can specify criteria for performing an untargeted mass analysis of product ions, while a sample is being introduced into the tandem mass spectrometer. For example, in an IDA method a precursor ion or mass spectrometry (MS) survey scan is performed to generate a precursor ion peak list. The user can select criteria to filter the peak list for a subset of the precursor ions on the peak list. MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other sample types, however, the complexity and dynamic range of compounds are very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a traditional DIA method, the actions of the tandem mass spectrometer are not varied among MS/MS scans based on data acquired in a previous precursor or product ion scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be very narrow so that the likelihood of multiple precursors within the window is small. This type of DIA method is called, for example, MS/MS^{ALL}. In an MS/MS^{ALL} method, a precursor ion mass selection window of about 1 amu is scanned or stepped across an entire mass range. A product ion spectrum is produced for each 1 amu precursor mass window. The time it takes to analyze or scan the entire mass range once is referred to as one scan cycle. Scanning a narrow precursor ion mass selection window across a wide precursor ion mass range during each cycle, however, is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 1-25 amu, or even larger. Like the MS/MS^{ALL} method, all the precursor ions in each precursor ion mass selection window are fragmented, and all of the product ions of all of the precursor ions in each mass selection window are mass analyzed. However, because a wider precursor ion mass selection window is used, the cycle time can be significantly reduced in comparison to the cycle time of the MS/MS^{ALL} method. Or, for liquid chromatography (LC), the accumulation time can be increased. Generally, for LC, the cycle time is defined by an LC peak. Enough points (intensities as a function of cycle time) must be obtained across an LC peak to determine its shape. When the cycle time is defined by the LC, the number of experiments or mass spectrometry scans that can be performed in a cycle defines how long each experiment or scan can accumulate ion observations. As a result, using a wider precursor ion mass selection window can increase the accumulation time.

U.S. Pat. No. 8,809,770 describes how SWATH acquisition can be used to provide quantitative and qualitative information about the precursor ions of compounds of interest. In particular, the product ions found from fragmenting a precursor ion mass selection window are compared to a database of known product ions of compounds of interest. In addition, ion traces or extracted ion chromatograms (XICs) of the product ions found from fragmenting a precursor ion mass selection window are analyzed to provide quantitative and qualitative information.

However, identifying compounds of interest in a sample analyzed using SWATH acquisition, for example, can be difficult. It can be difficult because either there is no precursor ion information provided with a precursor ion mass selection window to help determine the precursor ion that produces each product ion, or the precursor ion information provided is from a mass spectrometry (MS) observation that has a low sensitivity. In addition, because there is little or no specific precursor ion information provided with a precursor ion mass selection window, it is also difficult to determine if a product ion is convolved with or includes contributions from multiple precursor ions within the precursor ion mass selection window.

### Scanning SWATH Background

As a result, a method of scanning the precursor ion mass selection windows in SWATH acquisition, called scanning SWATH, was developed. Essentially, in scanning SWATH, a precursor ion mass selection window is scanned across a mass range so that successive windows have large areas of overlap and small areas of non-overlap. This scanning makes the resulting product ions a function of the scanned precursor ion mass selection windows. This additional information, in turn, can be used to identify the one or more precursor ions responsible for each product ion.

Scanning SWATH has been described in International Publication No. WO 2013/171459 A2 (hereinafter “the '459 Application”). In the '459 Application, a precursor ion mass selection window or precursor ion mass selection window of 25 Da is scanned with time such that the range of the precursor ion mass selection window changes with time. The timing at which product ions are detected is then correlated to the timing of the precursor ion mass selection window in which their precursor ions were transmitted.

The correlation is done by first plotting the mass-to-charge ratio (m/z) of each product ion detected as a function of the precursor ion m/z values transmitted by the quadrupole mass filter. Since the precursor ion mass selection window is scanned over time, the precursor ion m/z values transmitted by the quadrupole mass filter can also be thought of as times. The start and end times at which a particular product ion is detected are correlated to the start and end times at which its precursor is transmitted from the quadrupole. As a result, the start and end times of the product ion signals are used to determine the start and end times of their corresponding precursor ions.

Scanning SWATH has also been described in U.S. Pat. No. 10,068,753 (hereinafter “the '753 Patent”). The '753 Patent improves the accuracy of the correlation of product ions to their corresponding precursor ions by combining product ion spectra from successive groups of the overlapping rectangular precursor ion mass selection windows. Product ion spectra from successive groups are combined by successively summing the intensities of the product ions in the product ion spectra. This summing produces a function that can have a shape that is non-constant with precursor mass. The shape describes product ion intensity as a function of precursor mass. A precursor ion is identified from the function calculated for a product ion.

Systems and methods for identifying one or more precursor ions corresponding to a product ion in scanning SWATH data are further described in U.S. Pat. No. 10,651,019 (hereinafter “the '019 Patent”). Scanning SWATH is performed, producing a series of overlapping windows across the precursor ion mass range. Each overlapping window is fragmented and mass analyzed, producing a plurality of product ion spectra for the mass range. A product ion is selected from the spectra. Intensities for the selected product ion are retrieved for at least one scan across the mass range producing a trace of intensities versus precursor ion m/z. A matrix multiplication equation is created that describes how one or more precursor ions correspond to the trace for the selected production. The matrix multiplication equation is solved for one or more precursor ions corresponding to the selected product ion using a numerical method.

As described above, SWATH is a tandem mass spectrometry technique that allows a mass range to be scanned within a time interval using multiple precursor ion scans of adjacent or overlapping precursor ion mass selection windows. A mass filter selects each precursor mass window for fragmentation. A high-resolution mass analyzer is then used to detect the product ions produced from the fragmentation of each precursor mass window. SWATH allows the sensitivity of precursor ion scans to be increased without the traditional loss in specificity.

Unfortunately, however, the increased sensitivity that is gained through the use of sequential precursor mass windows in the SWATH method is not without cost. Each of these precursor mass windows can contain many other precursor ions, which confounds the identification of the correct precursor ion for a set of product ions. Essentially, the exact precursor ion for any given product ion can only be localized to a precursor mass window.

FIG. 2 is an exemplary plot 200 of a single precursor ion mass selection window that is typically used in a SWATH acquisition. Precursor ion mass selection window 210 transmits precursor ions with m/z values between M₁ and M₂, has set mass or center mass 215, and has sharp vertical edges 220 and 230. The SWATH precursor ion mass selection window width is M₂−M₁. The rate at which precursor ion mass selection window 210 transmits precursor ions is constant with respect to precursor m/z. Note that one skilled in the art can appreciate that the terms “m/z” and “mass” can be used interchangeably. The mass is easily obtained from the m/z value by multiplying the m/z value by the charge.

FIG. 3 is an exemplary series 300 of plots showing how product ions are correlated to precursor ions in conventional SWATH. Plot 310 shows a precursor ion mass range from 100 m/z to 300 m/z. When this precursor ion mass range is mass filtered and analyzed using a precursor ion scan, the precursor ion mass spectrum shown in plot 310 is found. The precursor ion mass spectrum includes precursor ion peaks 311, 312, 313, and 314, for example.

In conventional SWATH acquisition, a series of precursor ion mass selection windows, like precursor ion mass selection window 210 of FIG. 2, are selected across a precursor ion mass range. For example, ten precursor ion mass selection windows each of width 20 m/z can be selected for the precursor ion mass range from 100 m/z to 300 m/z shown in plot 310 of FIG. 3. Plot 320 shows three of the 10 precursor ion mass selection windows, 321, 322, and 323, for the precursor ion mass range from 100 m/z to 300 m/z. Note that the precursor ion mass selection windows of plot 320 do not overlap. In other conventional SWATH scans, the precursor ion mass selection windows can overlap.

For each conventional SWATH scan, the precursor ion mass selection windows are sequentially fragmented and mass analyzed. As a result, for each scan, a product ion spectrum is produced for each precursor ion mass selection window. Plot 331 is the product ion spectrum produced for precursor ion mass selection window 321 of plot 320. Plot 332 is the product ion spectrum produced for precursor ion mass selection window 322 of plot 320. And, plot 333 is the product ion spectrum produced for precursor ion mass selection window 323 of plot 320.

The product ions of a conventional SWATH are correlated to precursor ions by locating the precursor ion mass selection window of each product ion, and determining the precursor ions of the precursor ion mass selection window from the precursor ion spectrum obtained from a precursor ion scan. For example, product ions 341, 342, and 343 of plot 331 are produced by fragmenting precursor ion mass selection window 321 of plot 320. Based on its location in the precursor ion mass range and the results from a precursor ion scan, precursor ion mass selection window 321 is known to include precursor ion 311 of plot 310. Since precursor ion 311 is the only precursor ion in precursor ion mass selection window 321 of plot 320, product ions 341, 342, and 343 of plot 331 are correlated to precursor ion 311 of plot 310.

Similarly, product ion 361 of plot 333 is produced by fragmenting precursor ion mass selection window 323 of plot 320. Based on its location in the precursor ion mass range and the results from a precursor ion scan, precursor ion mass selection window 323 is known to include precursor ion 314 of plot 310. Since precursor ion 314 is the only precursor ion in precursor ion mass selection window 323 of plot 320, product ion 361 is correlated to precursor ion 314 of plot 310.

The correlation, however, becomes more difficult when a precursor ion mass selection window includes more than one precursor ion and those precursor ions may produce the same or a similar product ion. In other words, when interfering precursor ions occur in the same precursor ion mass selection window, it is not possible to correlate the common product ions to the interfering precursor ions without additional information.

For example, product ions 351 and 352 of plot 332 are produced by fragmenting precursor ion mass selection window 322 of plot 320. Based on its location in the precursor ion mass range and the results from a precursor ion scan, precursor ion mass selection window 322 is known to include precursor ions 312 and 313 of plot 310. As a result, product ions 351 and 352 of plot 332 can be from precursor ion 312 or 313 of plot 310. Further, precursor ions 312 and 313 may both be known to produce a product ion at or near the m/z of product ion 351. In other words, both precursor ions may provide contributions to product ion peak 351. As a result, the correlation of a product ion to a precursor ion or to a specific contribution from a precursor ion is made more difficult.

In conventional SWATH acquisition, chromatographic peaks, such as LC peaks, can also be used to improve the correlation. In other words, the compound of interest is separated over time and the SWATH acquisition is performed at a plurality of different elution or retention times. The retention times and/or the shapes of product and precursor ion chromatographic peaks are then compared to enhance the correlation. Unfortunately, however, because the sensitivity of the precursor ion scan is low, the chromatographic peaks of precursor ions may be convolved, further confounding the correlation.

In various embodiments, scanning SWATH provides additional information that is similar to that provided by chromatographic peaks, but with enhanced sensitivity. In scanning SWATH, overlapping precursor ion mass selection windows are used to correlate precursor and product ions. For example, a single precursor ion mass selection window such as precursor ion mass selection window 210 of FIG. 2 is shifted in small steps across a precursor mass range so that there is a large overlap between successive precursor ion mass selection windows. As the amount of overlap between precursor ion mass selection windows is increased, the accuracy in correlating the product ions to precursor ions is also increased.

Essentially, when the intensities of product ions produced from precursor ions filtered by the overlapping precursor ion mass selection windows are plotted as a function of the precursor ion mass selection window moving across the precursor mass range, each product ion has an intensity for the same precursor mass range that its precursor ion has been transmitted. In other words, for a rectangular precursor ion mass selection window (such as precursor ion mass selection window 210 of FIG. 2) that transmits precursor ions at a constant rate with respect to precursor mass, the edges (such as edges 220 and 230 of FIG. 2) define a unique boundary of both precursor ion precursor ion mass selection and product ion intensity as the precursor ion mass selection is stepped across the precursor mass range.

FIG. 4 is an exemplary plot 400 of a precursor ion mass selection window 410 that is shifted or scanned across a precursor ion mass range in order to produce overlapping precursor ion mass selection windows. Precursor ion mass selection window 410, for example, starts to transmit precursor ion with m/z value 420 when leading edge 430 reaches precursor ion with m/z value 420. As precursor ion mass selection window 410 is shifted across the m/z range, the precursor ion with m/z value 420 is transmitted until trailing edge 440 reaches m/z value 420.

When the intensities of the product ions from the product ion spectra produced by the overlapping windows are plotted, for example, as a function of the m/z value of leading edge 430, any product ion produced by the precursor ion with m/z value 420 would have an intensity between m/z value 420 and m/z value 450 of leading edge 430. One skilled in the art can appreciate that the intensities of the product ions produced by the overlapping windows can be plotted as a function of the precursor ion m/z value based on any parameter of precursor ion mass selection window 410 including, but not limited to, trailing edge 440, set mass, center of gravity, or leading edge 430.

FIG. 5 is an exemplary series 500 of plots showing how product ions are correlated to precursor ions in scanning SWATH. Plot 510 is the same as plot 310 of FIG. 3. Plot 510 of FIG. 5 shows a precursor ion mass range from 100 m/z to 300 m/z. When this precursor ion mass range is mass filtered and analyzed using a precursor ion scan, the precursor ion mass spectrum shown in plot 510 is found. The precursor ion mass spectrum includes precursor ion peaks 311, 312, 313, and 314, for example.

In scanning SWATH, however, rather than selecting and then fragmenting and mass analyzing non-overlapping precursor ion mass selection windows across the mass range, a precursor ion mass selection window is quickly moved or scanned across the precursor ion mass range with large overlaps between windows in each scanning SWATH scan. For example, during scan 1, precursor ion mass selection window 521 of plot 520 extends from 100 m/z to 120 m/z. The fragmentation of precursor ion mass selection window 521 and mass analysis of the resulting fragments during scan 1 produces the product ions of plot 531. Product ions 541, 542, and 543 of plot 531 are known to correlate to precursor ion 311 of plot 510, because precursor ion 311 is the only precursor within precursor ion mass selection window 521 of plot 520. Note that plot 531 includes the same product ions as plot 331 of FIG. 3.

For scan 2, precursor ion mass selection window 521 is shifted 1 m/z as shown in plot 530. Precursor ion mass selection window 521 of plot 530 no longer includes precursor ion 311 of plot 510. However, precursor ion mass selection window 521 of plot 530 now includes precursor ion 312 of plot 510. The fragmentation of precursor ion mass selection window 521 and mass analysis of the resulting fragments during scan 2 produces the product ion of plot 532. Product ion 551 of plot 532 is known to correlate to precursor ion 312 of plot 510, because precursor ion 312 is the only precursor within precursor ion mass selection window 521 of plot 530. Note that product ion 551 of plot 532 has the same m/z value as product ion 351 of plot 332 of FIG. 3, but a different intensity. From plot 532 of FIG. 5, it is now known what portion of 351 of plot 332 of FIG. 3 is from precursor ion 312 of plot 510.

For scan 3, precursor ion mass selection window 521 is shifted another 1 m/z as shown in plot 540. Precursor ion mass selection window 521 of plot 540 now includes precursor ions 312 and 313 of plot 510. The fragmentation of precursor ion mass selection window 521 and mass analysis of the resulting fragments during scan 3 produces the product ions of plot 533. Because precursor ion mass selection window 521 of plot 540 includes precursor ions 312 and 313 of plot 510, product ions 551 and 552 of plot 533 can be from either or both precursor ions.

Note that plot 533 includes the same product ions as plot 332 of FIG. 3. However, due to the additional information from scanning SWATH correlation is now possible. As mentioned above, from plot 532 of FIG. 5, it is now known what portion of 351 of plot 332 of FIG. 3 is from precursor ion 312 of plot 510. In other words, when the leading edges of precursor ion mass selection window 521 reaches precursor ion 312 of plot 510 and the trailing edges of precursor ion mass selection window 521 no longer includes precursor ion 312 of plot 510, the contribution of precursor ion 312 of plot 510 is known.

In addition, comparing plots 532 and 533 of FIG. 5 determines the contributions of precursor ion 313 of plot 510. Note that once the leading edge of precursor ion mass selection window 521 reaches precursor ion 313 of plot 510, product ion 552 of plot 533 appears and the intensity of product ion 551 increases. Thus product ion 552 is correlated to precursor ion 313 of plot 510 and the additional intensity of product ion 551 is also correlated to precursor ion 313 of plot 510.

### SUMMARY

A system, method, and computer program product are disclosed for extracting additional information from a DIA mass spectrometry experiment. The system includes an ion source device, a tandem mass spectrometer, and a processor.

The ion source device transforms a sample or compounds of interest from a sample into an ion beam. The tandem mass spectrometer divides a mass range of the ion beam into n precursor ion mass selections windows, and, for each window of the n windows, fragments precursor ions of each window and mass analyzes resulting product ions from the fragmentation. A product ion spectrum is produced for each window and n product ion spectra for the mass range.

The processor compares the n spectra to a library of product ion mass spectra for known compounds to identify an initial i compounds corresponding to l spectra. The processor performs a reinforcement learning algorithm using a number of steps. In step (a), acting as an agent of the RLA, the processor performs an action Aₜ that includes searching one or more compound databases for compounds related to the i compounds, producing j related compounds, and applying one or more deep learning prediction algorithms (DLPAs) to predict k product ion spectra for the i+j compounds. In step (b), acting as an environment of the RLA, the processor compares the k spectra to the n spectra, producing a state, Sₜ, in which i+j compounds produce m matching compounds and a reward, Rₜ, for the agent if m>i. In step (c), if the Rₜ is produced, the processor sets the i compounds to the m compounds and the/spectra to the k spectra, and repeats steps (a)-(c).

In some embodiments, a system for extracting additional information from a data independent acquisition (DIA) mass spectrometry experiment is provided, the system comprising: an ion source device that ionizes one or more compounds of a sample, producing an ion beam; a tandem mass spectrometer that divides a mass range of the ion beam into n precursor ion mass selections windows, and, for each window of the n windows, fragments precursor ions of each window and mass analyzes resulting product ions from the fragmentation, producing a product ion spectrum for each window and n product ion spectra for the mass range; and
a processor that compares the n spectra to a library of product ion mass spectra for known compounds to identify an initial i compounds corresponding to l spectra, and performs a reinforcement learning algorithm (RLA) in which the processor
(a) acting as an agent of the RLA, performs an action At that includes searching one or more compound databases for compounds related to the i compounds, producing j related compounds, and applying one or more deep learning prediction algorithms (DLPAs) to predict k product ion spectra for the i+j compounds, (b) acting as an environment of the RLA, compares the k spectra to the n spectra, producing a state, Sₜ, in which i+j compounds produce m matching compounds and a reward, Rₜ, for the agent if m>i, and (c) if the Rₜ is produced, sets the i compounds to the m compounds and the l spectra to the k spectra, and repeats steps (a)-(c).
   In some embodiments, a method for extracting additional information from a data independent acquisition (DIA) mass spectrometry experiment is provided, the method, comprising: instructing an ion source device to ionize one or more compounds of a sample using a processor, producing an ion beam; instructing a tandem mass spectrometer to divide a mass range of the ion beam into n precursor ion mass selections windows, and, for each window of the n windows, fragment precursor ions of each window and mass analyze resulting product ions from the fragmentation using the processor, producing a product ion spectrum for each window and n product ion spectra for the mass range; comparing the n product ion spectra to a library of product ion mass spectra for known compounds to identify an initial i compounds corresponding to l spectra of the sample using the processor, and performing a reinforcement learning algorithm (RLA) using the processor in which the processor acting (a) as an agent of the RLA, performs an action Aₜ that includes searching one or more compound databases for compounds related to the i compounds, producing j related compounds, and applying one or more deep learning prediction algorithms (DLPAs) to predict k product ion spectra for the i+j compounds, (b) acting as an environment of the RLA, compares the k spectra to the n spectra, producing a state, Sₜ, in which i+j compounds produce m matching compounds and a reward, Rₜ, for the agent if m>i, and (c) if the Rₜ is produced, sets the i compounds to the m compounds and the l spectra to the k spectra, and repeats steps (a)-(c).

In some embodiments, a computer program product, comprising a non-transitory tangible computer-readable storage medium whose contents include a program with instructions being executed on a processor for verifying compounds of a group detected by co-clustering are related to a biological process is provided, the computer program product comprising: providing a system, wherein the system comprises one or more distinct software modules, and wherein the distinct software modules comprise a control module and an analysis module; instructing an ion source device to ionizes one or more compounds of a sample using the control module, producing an ion beam; instructing a tandem mass spectrometer to divide a mass range of the ion beam into n precursor ion mass selections windows, and, for each window of the n windows, fragment precursor ions of each window and mass analyze resulting product ions from the fragmentation using the control module, producing a product ion spectrum for each window and n product ion spectra for the mass range; comparing the n product ion spectra to a library of product ion mass spectra for known compounds to identify an initial i compounds corresponding to l spectra using the analysis module, and performing a reinforcement learning algorithm (RLA) using the analysis module in which the analysis module (a) acting as an agent of the RLA, performs an action At that includes searching one or more compound databases for compounds related to the i compounds, producing j related compounds, and applying one or more deep learning prediction algorithms (DLPAs) to predict k product ion spectra for the i+j compounds, (b) acting as an environment of the RLA, compares the k spectra to the n spectra, producing a state, Sₜ, in which i+j compounds produce m matching compounds and a reward, Rₜ, for the agent if m>i, and (c) if the Rₜ is produced, sets the i compounds to the m compounds and the l spectra to the k spectra, and repeats steps (a)-(c).

In some embodiments, a system for extracting additional information from a data independent acquisition (DIA) mass spectrometry experiment is provided, the system comprising: a processor that receives from a tandem mass spectrometer, n product ion spectra, wherein the tandem mass spectrometer divides a mass range of an ion beam, from an ion source that ionizes one or more compounds of a sample, into n precursor ion mass selections windows, and, for each window of the n windows, fragments precursor ions of each window and mass analyzes resulting product ions from the fragmentation, producing a product ion spectrum for each window and the n product ion spectra for the mass range; compares the n spectra to a library of product ion mass spectra for known compounds to identify an initial i compounds corresponding to l spectra, and performs a reinforcement learning algorithm (RLA) in which the processor (a) acting as an agent of the RLA, performs an action At that includes searching one or more compound databases for compounds related to the i compounds, producing j related compounds, and applying one or more deep learning prediction algorithms (DLPAs) to predict k product ion spectra for the i+j compounds, (b) acting as an environment of the RLA, compares the k spectra to the n spectra, producing a state, Sₜ, in which i+j compounds produce m matching compounds and a reward, Rₜ, for the agent if m>i, and (c) if the Rₜ is produced, sets the i compounds to the m compounds and the l spectra to the k spectra, and repeats steps (a)-(c). In some embodiments, the processor receives from the tandem mass spectrometer, n×t product ion spectra, wherein the one or more compounds of the sample have been separated over time in a separation device and the ion source device has ionized the separated one or more compounds of the sample producing an ion beam and wherein the tandem mass spectrometer at each time step of t time steps, for each window of the n windows, fragments precursor ions of each window and mass analyzes resulting product ions from the fragmentation, producing a product ion spectrum for each window, n product ion spectra for the mass range, and n×t product ion spectra for the entire separation; compares the n×t spectra to the library of product ion mass spectra for known compounds to identify an initial i compounds corresponding to l spectra, and performs the RLA in which the processor (a) acting as an agent of the RLA, performs an action At that includes searching one or more compound databases for compounds related to the i compounds, producing j related compounds, and applying one or more deep learning prediction algorithms (DLPAs) to predict k product ion spectra for the i+j compounds, (b) acting as an environment of the RLA, compares the k spectra to the n×t spectra, producing a state, Sₜ, in which i+j compounds produce m matching compounds and a reward, Rₜ, for the agent if m>i, and (c) if the Rₜ is produced, sets the i compounds to the m compounds and the l spectra to the k spectra, and repeats steps (a)-(c).

In some embodiments, a computer program product is provided that comprises a non-transitory tangible computer-readable storage medium whose contents include a program with instructions being executed on a processor for verifying compounds of a group detected by co-clustering are related to a biological process, comprising: providing a system, wherein the system comprises one or more distinct software modules, and wherein the distinct software modules comprise an analysis module; the analysis module receiving from a tandem mass spectrometer, n product ion spectra, wherein the tandem mass spectrometer divides a mass range of an ion beam, from an ion source that ionizes one or more compounds of a sample, into n precursor ion mass selections windows, and, for each window of the n windows, fragments precursor ions of each window and mass analyzes resulting product ions from the fragmentation, producing a product ion spectrum for each window and the n product ion spectra for the mass range;
comparing the n product ion spectra to a library of product ion mass spectra for known compounds to identify an initial i compounds corresponding to l spectra using the analysis module, and performing a reinforcement learning algorithm (RLA) using the analysis module in which the analysis module (a) acting as an agent of the RLA, performs an action At that includes searching one or more compound databases for compounds related to the i compounds, producing j related compounds, and applying one or more deep learning prediction algorithms (DLPAs) to predict k product ion spectra for the i+j compounds, (b) acting as an environment of the RLA, compares the k spectra to the n spectra, producing a state, Sₜ, in which i+j compounds produce m matching compounds and a reward, Rₜ, for the agent if m>i, and (c) if the Rₜ is produced, sets the i compounds to the m compounds and the l spectra to the k spectra, and repeats steps (a)-(c).

In some embodiments, a system for extracting additional information from a data independent acquisition (DIA) mass spectrometry experiment is described. The system comprising: a processor that obtains n product ion spectra of one or more compounds of a sample; compares the n spectra to a library of product ion mass spectra for known compounds to identify an initial i compounds corresponding to l spectra, and performs a reinforcement learning algorithm (RLA) in which the processor (a) acting as an agent of the RLA, performs an action Aₜ that includes searching one or more compound databases for compounds related to the i compounds, producing j related compounds, and applying one or more deep learning prediction algorithms (DLPAs) to predict k product ion spectra for the i+j compounds, (b) acting as an environment of the RLA, compares the k spectra to the n spectra, producing a state, Sₜ, in which i+j compounds produce m matching compounds and a reward, Rₜ, for the agent if m>i, and (c) if the Rₜ is produced, sets the i compounds to the m compounds and the l spectra to the k spectra, and repeats steps (a)-(c).

In some embodiments, a method for extracting additional information from a data independent acquisition (DIA) mass spectrometry experiment is described. The method comprising: obtaining n product ion spectra in a processor; comparing the n product ion spectra to a library of product ion mass spectra for known compounds to identify an initial i compounds corresponding to l spectra of the sample using the processor, and performing a reinforcement learning algorithm (RLA) using the processor in which the processor (a) acting as an agent of the RLA, performs an action Aₜ that includes searching one or more compound databases for compounds related to the i compounds, producing j related compounds, and applying one or more deep learning prediction algorithms (DLPAs) to predict k product ion spectra for the i+j compounds, (b) acting as an environment of the RLA, compares the k spectra to the n spectra, producing a state, Sₜ, in which i+j compounds produce m matching compounds and a reward, Rₜ, for the agent if m>i, and (c) if the Rₜ is produced, sets the i compounds to the m compounds and the l spectra to the k spectra, and repeats steps (a)-(c).

In some embodiments, a computer program product, comprising a non-transitory tangible computer-readable storage medium whose contents include a program with instructions being executed on a processor for verifying compounds of a group detected by co-clustering are related to a biological process is described, comprising: providing a system, wherein the system comprises one or more distinct software modules, and wherein the distinct software modules comprise an analysis module; the analysis module obtaining n product ion spectra; comparing the n product ion spectra to a library of product ion mass spectra for known compounds to identify an initial i compounds corresponding to l spectra using the analysis module, and performing a reinforcement learning algorithm (RLA) using the analysis module in which the analysis module (a) acting as an agent of the RLA, performs an action Aₜ that includes searching one or more compound databases for compounds related to the i compounds, producing j related compounds, and applying one or more deep learning prediction algorithms (DLPAs) to predict k product ion spectra for the i+j compounds, (b) acting as an environment of the RLA, compares the k spectra to the n spectra, producing a state, Sₜ, in which i+j compounds produce m matching compounds and a reward, Rₜ, for the agent if m>i, and (c) if the Rₜ is produced, sets the i compounds to the m compounds and the l spectra to the k spectra, and repeats steps (a)-(c).

These and other features of the applicant's teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary plot of a single precursor ion mass selection window that is typically used in a SWATH acquisition.

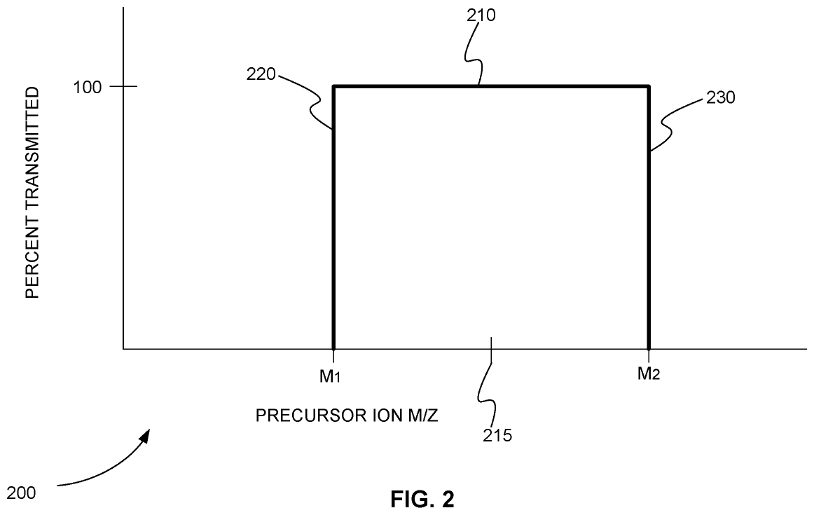

FIG. 3 is an exemplary series 3 of plots showing how product ions are correlated to precursor ions in conventional SWATH.

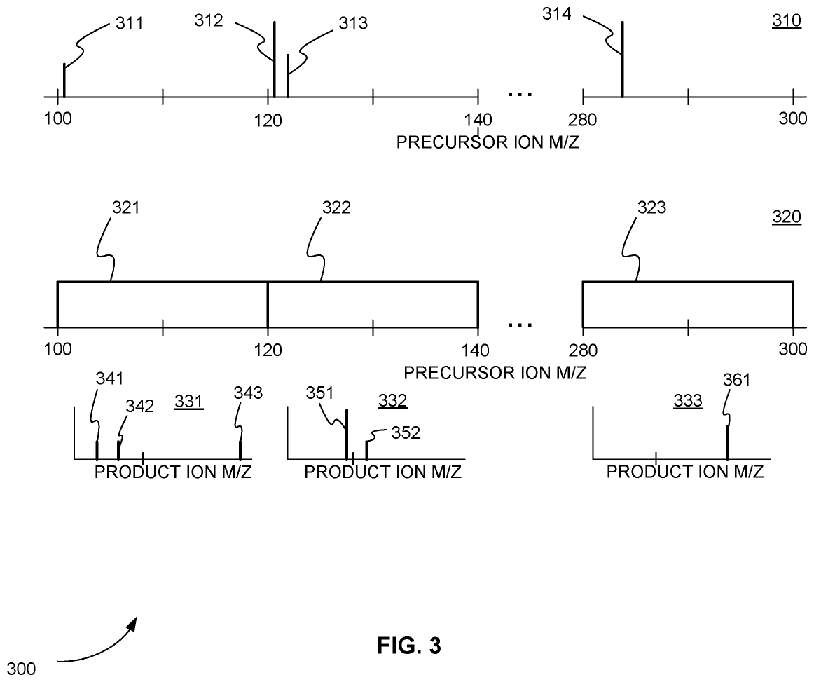

FIG. 4 is an exemplary plot of a precursor ion mass selection window that is shifted or scanned across a precursor ion mass range in order to produce overlapping precursor ion mass selection windows.

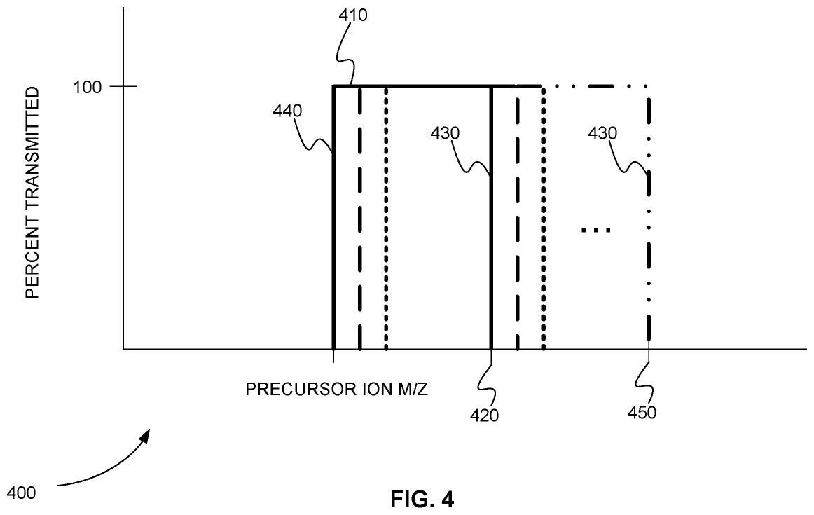

FIG. 5 is an exemplary series of plots showing how product ions are correlated to precursor ions in scanning SWATH.

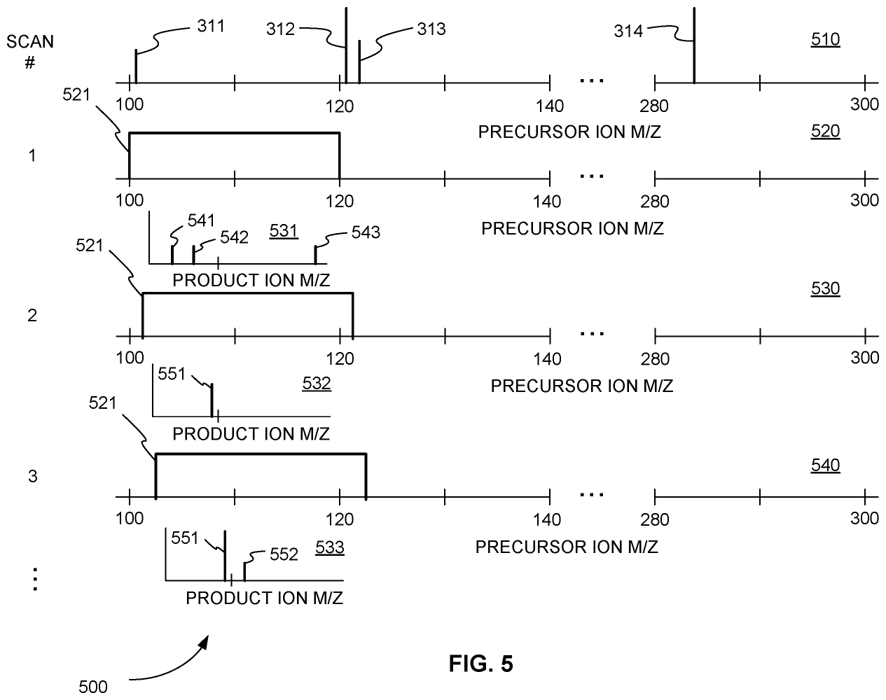

FIG. 6 is an exemplary diagram of the method of the Ronghui Paper.

FIG. 7 is an exemplary diagram showing the components of a reinforcement learning algorithm.

FIG. 8 is an exemplary diagram showing how a reinforcement learning algorithm is used to maximize the number of peptides identified in experimental DIA data obtained for a sample, in accordance with various embodiments.

FIG. 9 is a schematic diagram showing a mass spectrometry system for extracting additional information from a DIA mass spectrometry experiment, in accordance with various embodiments.

FIG. 10 is a flowchart showing a method for extracting additional information from a DIA mass spectrometry experiment, in accordance with various embodiments.

FIG. 11 is a schematic diagram of a system that includes one or more distinct software modules that performs a method for extracting additional information from a DIA mass spectrometry experiment, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

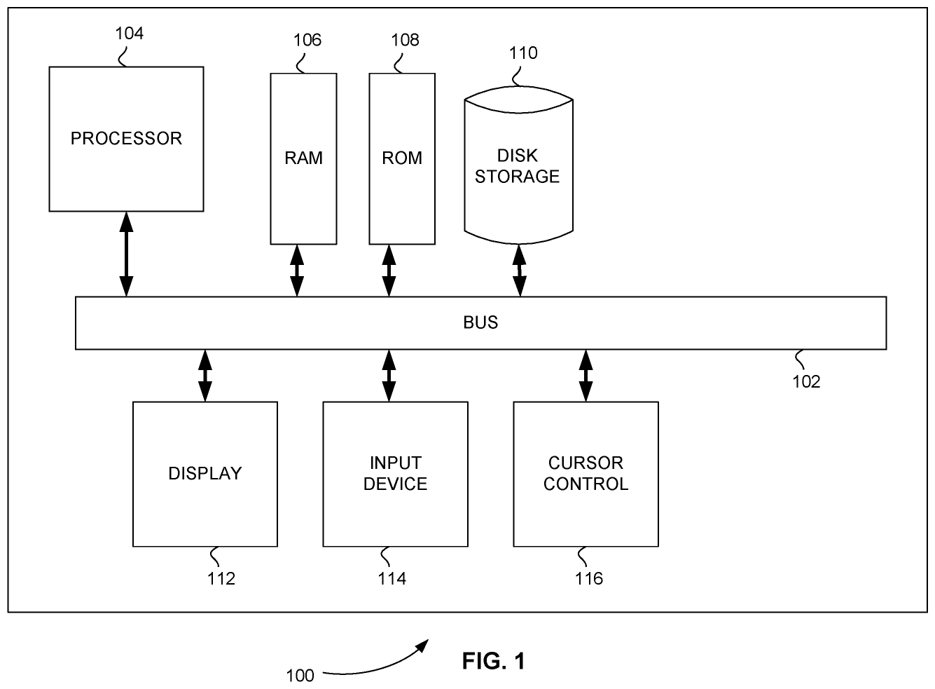

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein. Alternatively, hard-wired circuitry may be used in place of or in combination with software instructions to implement the present teachings. Thus implementations of the present teachings are not limited to any specific combination of hardware circuitry and software.

The term “computer-readable medium” as used herein refers to any media that participates in providing instructions to processor 104 for execution. Such a medium may take many forms, including but not limited to, non-volatile media, volatile media, and precursor ion mass selection media. Non-volatile media includes, for example, optical or magnetic disks, such as storage device 110. Volatile media includes dynamic memory, such as memory 106. Precursor ion mass selection media includes coaxial cables, copper wire, and fiber optics, including the wires that comprise bus 102.

Common forms of computer-readable media include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone or in certain embodiments in software alone. The present teachings may be implemented with both object-oriented and non-object-oriented programming systems.

### Using Reinforcement Learning in DIA Data Extraction

As described above, DIA data is very information-rich and libraries used to extract information from DIA data can come from a range of different sources. Recently, deep learning methods have been used to predict peptide spectra. Although promising, the use of libraries created with deep learning methods have increased the false-negative rate of peptide identifications and increased the overall computational time required for peptide identifications.

As a result, there is a need for systems and methods that can allow deep learning prediction methods to be used to extract information from DIA data without producing a large amount of false-negative results and without significantly increasing the computation time required. The increase in the FDR rate is a result of the increased complexity of convolution in mass space and the increase in extraction of compounds which are not present in the data.

In various embodiments, current data workflows are used to identify proteins and other compounds that may be changing in a significant manner in relation to experimental data. In-silico fragmentation of this list of proteins and other compounds provides input for a deep learning algorithm, for example, that can, in turn, provide both additional spectra and retention times (RTs). This is then used to reanalyze the DIA data and the process is repeated as needed.

Additionally, a reinforcement learning pattern can be applied on top of the deep learning systems. In this reinforcement learning, the original library produced from DDA data is used to refine the library to the instrument conditions that are being used and enhance the confidence in the predictions of the model. It is also possible to reuse the intensity information for compounds extracted from the SWATH data to reconstruct the MSMS fragmentation spectra and these intern be used in the reinforcement learning.

In other words, various embodiments address the issue of brute-force spectral library approaches when using FDR estimation, which inherently assumes a large proportion of the library exists in the sample. This results in the large false negative rates on larger libraries as opposed to smaller libraries tailored to the sample. In addition, various embodiments aim to expand the pre-existing library to include proteins that have low sequence coverage and may be changing in a significant manner in relation to the experimental metadata. This increases proteome coverage.

Deep learning methods like ProSIT, pDeep3, and MS2PIP have proven that deep learning can effectively be used to predict fragment intensities and RTs for proteins that were not used during training. These models can be trained to include experimental conditions and instrument type.

For example, Ronghui et al., “Hybrid Spectral Library Combining DIA-MS Data and a Targeted Virtual Library Substantially Deepens the Proteome Coverage,” iScience, Volume 23, Issue 3, 2020, 100903, ISSN 2589-0042, https://doi.org/10.1016/j.isci.2020.100903, (hereinafter the “Ronghui Paper”) show that extending a library using a targeted sub-proteome virtual library increases the number of proteins identified.

The Ronghui Paper builds a hybrid spectral library that combines an experimental library with a protein family-targeted virtual predicted library through deep learning (pDeep and DeepRT). The Ronghui Paper also mentions that predicting all peptides of entire proteomes results in large libraries and increases false discovery rates. Since biological studies focus on specific protein classes, the Ronghui Paper recommends building targeted virtual libraries for a given protein superfamily.

Various embodiments described herein differ from the Ronghui Paper in the strategy used to predict related compounds. Various embodiments described herein also differ from the Ronghui Paper by using reinforcement learning to iteratively improve on prediction models with new data.

Various embodiments described herein expand spectral libraries with additional predicted spectra which may not already exist in the original libraries used. As opposed to a brute force prediction of all possible theoretical compounds, these embodiments provide a more focused approach in which libraries are enhanced only with related proteins or compounds for the target experiment. These new enhanced libraries provide a deeper coverage of proteins or pathways of quantitative interest. In addition, iterative learning improves the prediction models as new results are generated.

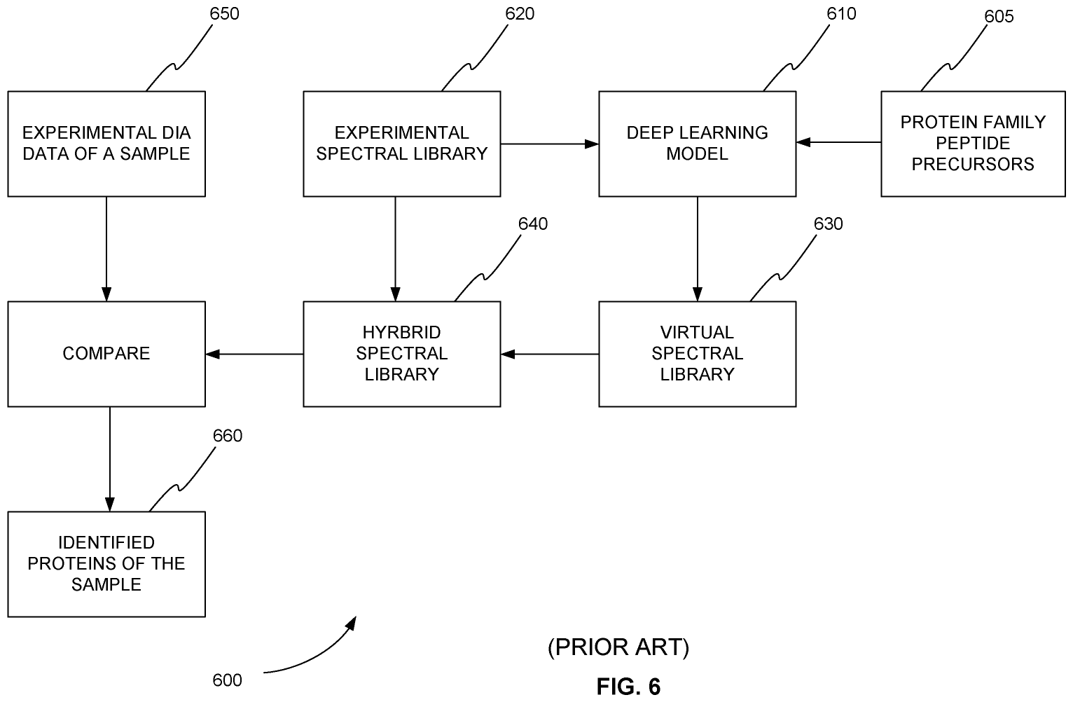

FIG. 6 is an exemplary diagram 600 of the method of the Ronghui Paper. Initially, a targeted protein family is in-silico digested producing a set of peptide precursors 605. Set of peptide precursors 605 is provided as input to pre-trained deep learning model 610. Essentially, deep learning models like pDeep and DeepRT predict fragment ion intensities and retention times, respectively, from peptide precursors 605 (or peptide sequences). Spectral library 620 for a mass spectrometry experiment includes actual experimental spectra produced for a set of known compounds or proteins by a specific mass spectrometer, using a DDA method for example. Using transfer learning, spectral library 620 is used to re-train deep learning model 610 producing a re-trained model.

Re-trained deep learning model 610 is then used to produce virtual spectral library 630 for the targeted protein family. Spectral library 620 and virtual spectral library 630 are then combined to produce hybrid spectral library 640.

Finally, experimental DIA data 650 of a sample is compared to hybrid spectral library 640 to identify proteins 660 found in the sample.

As shown in FIG. 6, the method of the Ronghui Paper uses spectral library 620 to re-train deep learning model 610 and also combines spectral library 620 with virtual spectral library 630 to produce hybrid spectral library 640. The Ronghui Paper does not, however, directly use peptides digested in silico to produce additional virtual spectra, does not iteratively update inputs to deep learning model 610, and does not perform reinforcement learning.

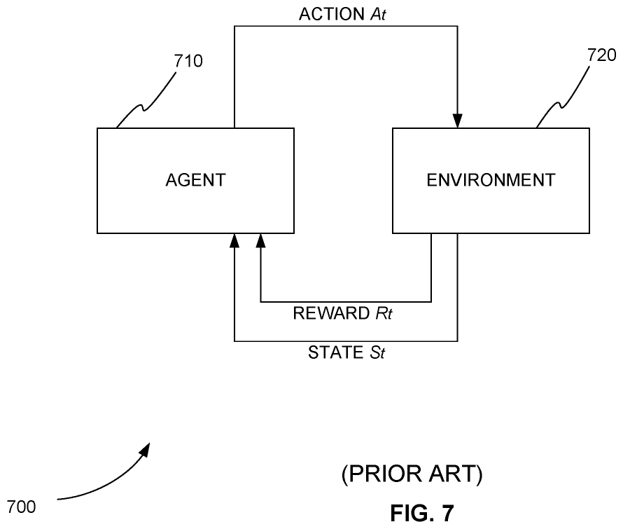

FIG. 7 is an exemplary diagram 700 showing the components of a reinforcement learning algorithm. Reinforcement learning involves interactions between an agent 710 and an environment 720. Agent 710 performs an action, Aᵢ, with respect to Environment 720. As a result of Aᵢ, agent 710 is in a state, Sᵢ. Agent 710 also receives a reward, Rᵢ, for Aᵢ. Rewards can also include punishments. Interactions between agent 710 and environment 720 continue until the cumulative rewards or punishments received by Agent 710 exceed some threshold, for example.

In various embodiments, the identification of compounds from DIA data is a reinforcement learning problem in which previous compound identifications are used to predict additional compound identifications. In this case, agent 710 is an algorithm trying to identify a maximum number of compounds in experimental DIA data of a sample. Environment 720 is the extraction of compounds from the experimental DIA data or, more specifically, a comparison of the experimental DIA data of a sample with virtual spectra produced by a deep learning algorithm.

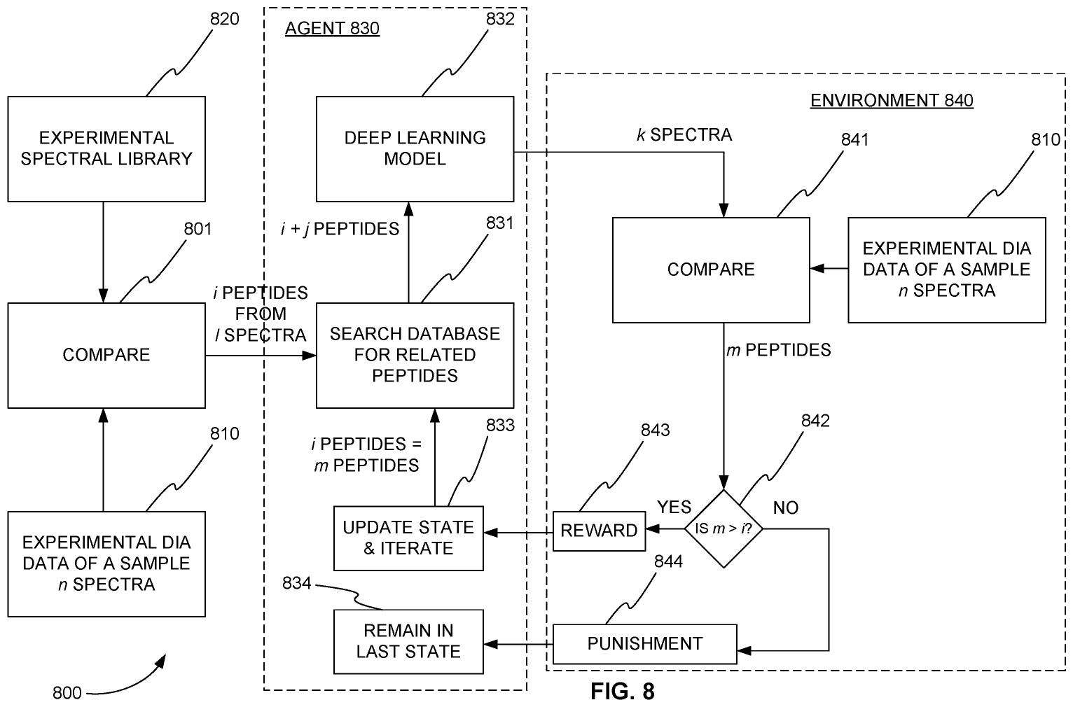

FIG. 8 is an exemplary diagram 800 showing how a reinforcement learning algorithm is used to maximize the number of peptides identified in experimental DIA data obtained for a sample, in accordance with various embodiments. Initially, a comparison 801 is performed in which n product ion spectra of experimental DIA data 810 of the sample are compared to an experimental spectral library 820 that includes spectra corresponding to a number of different known compounds. From comparison 801, i matching peptides are found corresponding to l spectra.

The i peptides and l spectra are provided to agent 830 of the reinforcement learning algorithm as the initial state of agent 830. In other words, the identification of i peptides and l spectra of a library is the initial state of agent 830 from experimental DIA data 810.

Agent 830 performs search 831 of a peptide database using the i peptides to find j related peptides. Searching for related peptides is well known to one of skill in the art and can be accomplished in many different ways. For example, Bimpikis et al., BLAST2SRS, a web server for flexible retrieval of related protein sequences in the SWISS-PROT and SPTrEMBL databases, Nucleic Acids Res, 2003 Jul. 1; 31(13):3792-4, (hereinafter the “Bimpikis Paper”) describe using peptide databases, such as SWISS-PROT and SPTrEMBL, to find related peptides. In the Bimpikis Paper, peptide databases are searched using a peptide sequence or a keyword related to a peptide. In various embodiments, a search can also include a retention time of a peptide. Note that one of skill in the art also understands that various embodiments described herein in regard to peptides equally apply to proteins.

The SWISS-PROT and SPTrEMBL databases have been combined under a single database called the UniProt database. As a result, search 831 can use the UniProt database to find the j related peptides, for example.

In order to produce virtual or theoretical spectra for the j peptides, agent 830 uses deep learning model 832. Deep learning model 832 of a deep learning algorithm can produce product ion spectra for the j peptides and these spectra can be combined with the l spectra of experimental spectral library 820 corresponding to the i peptides, producing a hybrid virtual library, like that of the Ronghui Paper. Alternatively and as shown in FIG. 8, the j peptides can be combined with the i peptides. Deep learning model 832 then produces k virtual product ion spectra for the i+j peptides.

The action of agent 830 is, therefore, to provide k spectra for environment 840. Environment 840 performs comparison 841 of k spectra with the n spectra of experimental DIA data 810, producing m matching peptides.

The goal of the reinforcement learning algorithm is to maximize the number of peptides identified in experimental DIA data 810. As a result, environment 840 makes a decision 842 regarding the m peptides found from comparison 841. Environment 840 determines if the number of peptides identified is increased by comparing the number of peptides identified currently, m, with the number of peptides identified previously, i.

If m>i, the number of peptides identified by the reinforcement learning algorithm is still increasing. As a result, environment 840 provides reward 843 to agent 830. Upon receiving reward 843, agent 830 performs an update 833 of its state and starts another iteration of the reinforcement learning algorithm. Update 833 includes setting or resetting the i peptides to be the m peptides and the l spectra to be the k spectra.

If m≤i, the number of peptides identified by the reinforcement learning algorithm is no longer increasing. As a result, environment 840 provides punishment 844 to agent 830. Upon receiving punishment 844, agent 830 performs an update 834 of its state and ends the reinforcement learning algorithm. Update 834 includes identifying the peptides of experimental DIA data 810 as the previously identified i peptides and identifying the virtual library of experimental DIA data 810 to include the previously identified l spectra.

In contrast to the method of the Ronghui Paper shown in FIG. 6, the method of FIG. 8 expands the number of identifications by finding compounds related to the previously identified compounds. Because an entire protein family is not used to expand the number of identifications, as in the method of the Ronghui Paper, the FDR is improved over the method of the Ronghui Paper. Because the number of compounds related to the previously identified compounds is generally much smaller than the number of compounds in a protein family, the computational time required for compound identification is reduced in comparison to the method of the Ronghui Paper.

### System for Extracting Additional Information

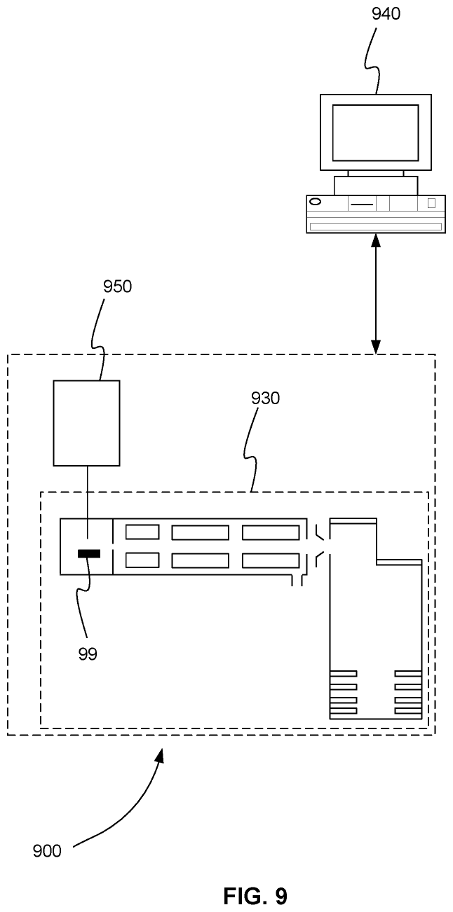

FIG. 9 is a schematic diagram 900 showing a mass spectrometry system for extracting additional information from a DIA mass spectrometry experiment, in accordance with various embodiments. System 900 of FIG. 9 includes ion source device 910, tandem mass spectrometer 930, and processor 940. In various embodiments, ion source device 910 can be part of tandem mass spectrometer 930 or a separate device.

In various embodiments, system 900 can further include sample introduction device 950. Sample introduction device 950 introduces one or more compounds of interest from a sample to ion source device 910 over time, for example. Sample introduction device 950 can perform techniques that include, but are not limited to, injection, liquid chromatography, gas chromatography, capillary electrophoresis, or ion mobility.

Ion source device 910 transforms a sample or compounds of interest from a sample provided by sample introduction device 950 into an ion beam, for example. Ion source device 910 can perform ionization techniques that include, but are not limited to, matrix assisted laser desorption/ionization (MALDI) or electrospray ionization (ESI).

Tandem mass spectrometer 930 divides a mass range of the ion beam into n precursor ion mass selections windows, and, for each window of the n windows, fragments precursor ions of each window and mass analyzes resulting product ions from the fragmentation. A product ion spectrum is produced for each window and n product ion spectra for the mass range.

Processor 940 can be, but is not limited to, a computer, a microprocessor, the computer system of FIG. 1, or any device capable of sending and receiving control signals and data to and from tandem mass spectrometer 930 and processing data. Processor 940 is in communication with ion source device 910 and tandem mass spectrometer 930.

Processor 940 compares the n spectra to a library of product ion mass spectra for known compounds to identify an initial i compounds corresponding to l spectra. Processor 940 performs a reinforcement learning algorithm using a number of steps. In step (a), acting as an agent of the RLA, processor 940 performs an action Aₜ that includes searching one or more compound databases for compounds related to the i compounds, producing j related compounds, and applying one or more deep learning prediction algorithms (DLPAs) to predict k product ion spectra for the i+j compounds. In step (b), acting as an environment of the RLA, processor 940 compares the k spectra to the n spectra, producing a state, Sₜ, in which i+j compounds produce m matching compounds and a reward, Rₜ, for the agent if m>i. In step (c), if the Rₜ is produced, processor 940 sets the i compounds to the m compounds and the l spectra to the k spectra, and repeats steps (a)-(c).

In various embodiments, system 900 further includes separation device 950 that separates the one or more compounds of the sample over time. As a result, n×t production spectra are produced for the entire separation. Processor 940 compares the n×t spectra to the library of product ion mass spectra for known compounds to identify an initial i compounds corresponding to l spectra. In step (b), acting as an environment of the RLA, processor 940 compares the k spectra to the n×t spectra, producing a state, Sₜ, in which i+j compounds produce m matching compounds and a reward, Rₜ, for the agent if m>i.

In various embodiments, processor 940 compares the n×t product ion spectra and retention times derived from the n×t product ion spectra to the library of product ion mass spectra and in step (b) the predicted spectra and retention times for the i+j compounds are compared to the n×t product ion spectra and retention times derived from the n×t product ion spectra.

In various embodiments, processor 940 further re-trains the one or more DLPAs using the i compounds and the corresponding to l spectra found from the comparison of the n spectra to the library before steps (a)-(c).

In various embodiments, the l spectra found from the comparison of the n spectra to the library include one or more of the matching spectra of the n spectra and the matching spectra of the library. In other words, the l spectra can be from the DIA data, the library, or both. The DIA data can also include XICs of the ion intensity measurements, the areas of those XICs, or the centroids of those XICs.

In various embodiments, the one or more compounds of the sample include one or more peptides, the library includes a library of product ion mass spectra for known peptides, the i compounds include i peptides, the i compounds include i peptides, the m compounds include m peptides, the one or more compound databases include one or more peptide databases.

In various embodiments, in step (a) processor 940 searches one or more peptide databases for peptides related to at least one peptide of the i peptides using a sequence, a keyword, or a retention time of the at least one peptide.

In various embodiments, the one or more peptide databases include UniProt.

In various embodiments, the one or more DLPAs include one or more of ProSIT, pDeep, pDeep3, DeepRT, and MS2PIP.

In various embodiments, in step (b), processor 940 further produces a punishment, Pₜ, for the agent if m≤i.

In various embodiments, in step (c), if the Pₜ is produced, processor 940 identifies the i compounds as the compounds found in the sample and l spectra as the spectra of a virtual library for the sample.

### Method for Extracting Additional Information

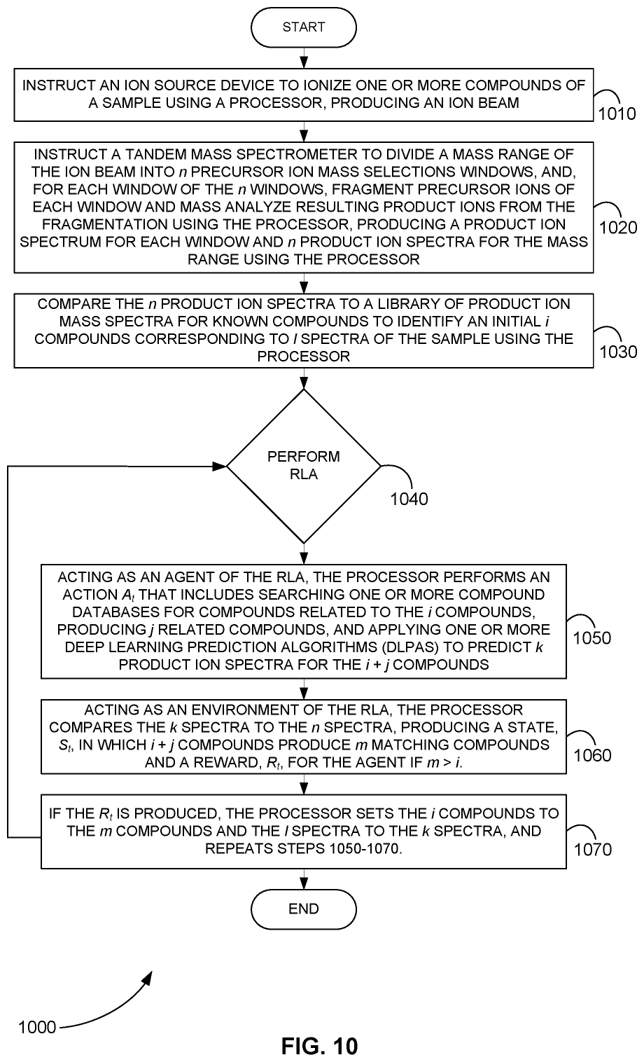

FIG. 10 is a flowchart 1000 showing a method for extracting additional information from a DIA mass spectrometry experiment, in accordance with various embodiments.

In step 1010 of method 1000, an ion source device is instructed to ionize one or more compounds of a sample using a processor, producing an ion beam.

In step 1020, a tandem mass spectrometer is instructed to divide a mass range of the ion beam into n precursor ion mass selections windows, and, for each window of the n windows, fragment precursor ions of each window and mass analyze resulting product ions from the fragmentation using the processor, producing a product ion spectrum for each window and n product ion spectra for the mass range using the processor.

In step 1030, the n product ion spectra are compared to a library of product ion mass spectra for known compounds to identify an initial i compounds corresponding to l spectra of the sample using the processor.

In step 1040, a reinforcement learning algorithm (RLA) is performed using the processor in which the processor performs the following steps.

In step 1050, acting as an agent of the RLA, the processor performs an action Aₜ that includes searching one or more compound databases for compounds related to the i compounds, producing j related compounds, and applying one or more deep learning prediction algorithms (DLPAs) to predict k product ion spectra for the i+j compounds.

In step 1060, acting as an environment of the RLA, the processor compares the k spectra to the n spectra, producing a state, Sₜ, in which i+j compounds produce m matching compounds and a reward, Rₜ, for the agent if m>i.

In step 1070, if the Rₜ is produced, the processor sets the i compounds to the m compounds and the l spectra to the k spectra, and repeats steps 1050-1070.

### Computer Program Product for Extracting Additional Information

In various embodiments, a computer program product includes a non-transitory tangible computer-readable storage medium whose contents include a program with instructions being executed on a processor so as to extract additional information from a DIA mass spectrometry experiment. This method is performed by a system that includes one or more distinct software modules.

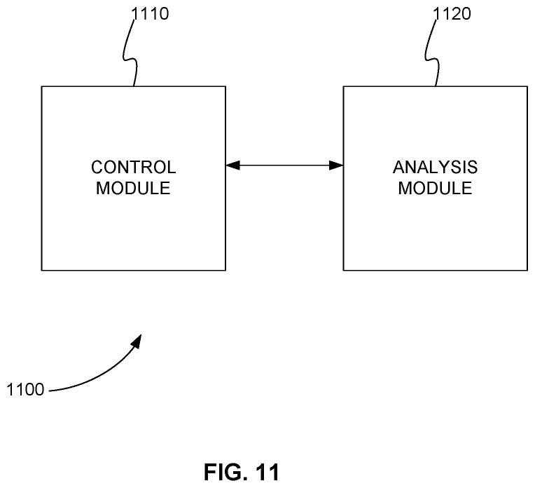

FIG. 11 is a schematic diagram of a system 1100 that includes one or more distinct software modules that performs a method for extracting additional information from a DIA mass spectrometry experiment, in accordance with various embodiments. System 1100 includes control module 1110 and analysis module 1120.

Control module 1110 instructs an ion source device to ionize one or more compounds of a sample, producing an ion beam. Control module 1410 a tandem mass spectrometer to divide a mass range of the ion beam into n precursor ion mass selections windows, and, for each window of the n windows, fragment precursor ions of each window and mass analyze resulting product ions from the fragmentation, producing a product ion spectrum for each window and n product ion spectra for the mass range.

Analysis module 1120 compares the n product ion spectra to a library of product ion mass spectra for known compounds to identify an initial i compounds corresponding to l spectra. Analysis module 1120 performs a reinforcement learning algorithm (RLA) in which analysis module 1120 performs a number of steps.

The control module and analysis module need not be present in the same computer program product and they may be separated into different computer program products that are executed on different processors. In certain embodiments, a computer program product comprising the control module may be executed to acquire data from a tandem mass spectrometer and the data stored and/or transferred to a separate computer program product comprising the analysis module to perform the steps as described herein. In certain embodiments, a software product comprising the analysis module on its own can be utilized to process the data using the within teachings by receiving data acquired from the tandem mass spectrometer.

In step (a), acting as an agent of the RLA, analysis module 1120 performs an action Aₜ that includes searching one or more compound databases for compounds related to the i compounds, producing j related compounds, and applying one or more deep learning prediction algorithms (DLPAs) to predict k product ion spectra for the i+j compounds.

In step (b), acting as an environment of the RLA, analysis module 1120 compares the k spectra to the n spectra, producing a state, Sₜ, in which i+j compounds produce m matching compounds and a reward, Rₜ, for the agent if m>i.

In step (c), if the Rₜ is produced, analysis module 1120 sets the i compounds to the m compounds and the l spectra to the k spectra, and repeats steps (a)-(c).

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

1. A system for extracting additional information from a data independent acquisition (DIA) mass spectrometry experiment, comprising:
a processor that
obtains n product ion spectra of one or more compounds of a sample;
compares the n spectra to a library of product ion mass spectra for known compounds to identify an initial i compounds corresponding to l spectra, and performs a reinforcement learning algorithm (RLA) in which the processor
a. acting as an agent of the RLA, performs an action Aₜ that includes searching one or more compound databases for compounds related to the i compounds, producing j related compounds, and applying one or more deep learning prediction algorithms (DLPAs) to predict k product ion spectra for the i+j compounds,
b. acting as an environment of the RLA, compares the k spectra to the n spectra, producing a state, Sₜ, in which i+j compounds produce m matching compounds and a reward, Rₜ, for the agent if m>i, and
c. if the Rₜ is produced, sets the i compounds to the m compounds and the l spectra to the k spectra, and repeats steps (a)-(c).

2. The system of claim 1, wherein
the processor
obtains n×t product ion spectra, wherein the one or more compounds of the sample were separated over time in a separation device and an ion source device has ionized the separated one or more compounds of the sample producing an ion beam and wherein a tandem mass spectrometer has divided a mass range of the ion beam into n precursor ion mass selection windows and where at each time step of t time steps, for each window of the n windows, fragments precursor ions of each window and mass analyzes resulting product ions from the fragmentation, producing a product ion spectrum for each window, n product ion spectra for the mass range, and n×t product ion spectra for the entire separation;
compares the n×t spectra to the library of product ion mass spectra for known compounds to identify an initial i compounds corresponding to l spectra, and
performs the RLA in which the processor
a. acting as an agent of the RLA, performs an action Aₜ that includes searching one or more compound databases for compounds related to the i compounds, producing j related compounds, and applying one or more deep learning prediction algorithms (DLPAs) to predict k product ion spectra for the i+j compounds,
b. acting as an environment of the RLA, compares the k spectra to the n×t spectra, producing a state, Sₜ, in which i+j compounds produce m matching compounds and a reward, Rₜ, for the agent if m>i, and
c. if the Rₜ is produced, sets the i compounds to the m compounds and the l spectra to the k spectra, and repeats steps (a)-(c).

3. The system of claim 1, wherein processor compares the n×t product ion spectra and retention times derived from the n×t product ion spectra to the library of product ion mass spectra and in step (b) the predicted spectra and retention times for the i+j compounds are compared to the n×t product ion spectra and retention times derived from the n×t production spectra.

4. The system of claim 1, wherein the processor further re-trains the one or more DLPAs using the i compounds and the corresponding to l spectra found from the comparison of the n spectra to the library before steps (a)-(c) the library before steps (a)-(c).

5. The system of claim 1, wherein the one or more compounds of the sample comprise one or more peptides, the library comprises a library of product ion mass spectra for known peptides, the i compounds comprise i peptides, the i compounds comprise i peptides, the m compounds comprise m peptides, the one or more compound databases comprise one or more peptide databases.

6. The system of claim 1, wherein in step (a) the processor searches one or more peptide databases for peptides related to at least one peptide of the i peptides using a sequence of the at least one peptide.

7. The system of claim 1, wherein in step (a) the processor searches one or more peptide databases for peptides related to at least one peptide of the i peptides using a keyword of the at least one peptide.

8. The system of claim 1, wherein in step (a) the processor searches one or more peptide databases for peptides related to at least one peptide of the i peptides using a retention time of the at least one peptide.

9. The system of claim 1, wherein the one or more peptide databases comprise UniProt.

10. The system of claim 1, wherein the l spectra found from the comparison of the n spectra to the library include one or more of the matching spectra of the n spectra and the matching spectra of the library.

11. The system of claim 1, wherein the one or more DLPAs comprise one or more of ProSIT, pDeep, pDeep3, DeepRT, and MS2PIP.

12. The system of claim 1, wherein, in step (b), the processor further produces a punishment, Pₜ, for the agent if m≤i.

13. The system of claim 1, wherein, in step (c), if the Pₜ is produced, the processor identifies the i compounds as the compounds found in the sample and l spectra as the spectra of a virtual library for the sample.

14. A method for extracting additional information from a data independent acquisition (DIA) mass spectrometry experiment, comprising:
obtaining n product ion spectra in a processor;
comparing the n product ion spectra to a library of product ion mass spectra for known compounds to identify an initial i compounds corresponding to l spectra of the sample using the processor, and
performing a reinforcement learning algorithm (RLA) using the processor in which the processor
a. acting as an agent of the RLA, performs an action Aₜ that includes searching one or more compound databases for compounds related to the i compounds, producing j related compounds, and applying one or more deep learning prediction algorithms (DLPAs) to predict k product ion spectra for the i+j compounds,
b. acting as an environment of the RLA, compares the k spectra to the n spectra, producing a state, Sₜ, in which i+j compounds produce m matching compounds and a reward, Rₜ, for the agent if m>i, and
c. if the Rₜ is produced, sets the i compounds to the m compounds and the l spectra to the k spectra, and repeats steps (a)-(c).

15. A computer program product, comprising a non-transitory tangible computer-readable storage medium whose contents include a program with instructions being executed on a processor for verifying compounds of a group detected by co-clustering are related to a biological process, comprising:
providing a system, wherein the system comprises one or more distinct software modules, and wherein the distinct software modules comprise an analysis module;
the analysis module obtaining n product ion spectra;
comparing the n product ion spectra to a library of product ion mass spectra for known compounds to identify an initial i compounds corresponding to l spectra using the analysis module, and
performing a reinforcement learning algorithm (RLA) using the analysis module in which the analysis module
a. acting as an agent of the RLA, performs an action Aₜ that includes searching one or more compound databases for compounds related to the i compounds, producing j related compounds, and applying one or more deep learning prediction algorithms (DLPAs) to predict k product ion spectra for the i+j compounds,
b. acting as an environment of the RLA, compares the k spectra to the n spectra, producing a state, Sₜ, in which i+j compounds produce m matching compounds and a reward, Rₜ, for the agent if m>i, and
c. if the Rₜ is produced, sets the i compounds to the m compounds and the l spectra to the k spectra, and repeats steps (a)-(c).

16. The system of claim 1 wherein the n product ion spectra are obtained from a tandem mass spectrometer, wherein the tandem mass spectrometer divides a mass range of an ion beam, from an ion source that ionizes one or more compounds of a sample, into n precursor ion mass selections windows, and, for each window of the n windows, fragments precursor ions of each window and mass analyzes resulting product ions from the fragmentation, producing a product ion spectrum for each window and the n product ion spectra for the mass range.

17. The method of claim 14 wherein the method includes instructing an ion source to ionize one or more compounds of a sample using the processor, producing an ion beam; instructing a tandem mass spectrometer to divide a mass range of the ion beam into n precursor ion mass selection windows, and, for each window of the n windows, fragment precursor ions of each window and mass analyze resulting product ions from the fragmentation using the processor, producing a product ion spectrum for each window and the n product ion spectra for the mass range.
